(19) United States
(12) Patent Application Publication
(10) Pub. No.: US 2003/0083313 A1
(43) Pub. Date: May 1, 2003

(54) Means of tumor therapy

(76) Inventors: Reinhard Zeisig, Berlin (DE); Iduna Fichtner, Berlin (DE); Dietrich Arndt, Berlin (DE)

Correspondence Address: GABRIEL P. KATONA, GOODWIN PROCTER L.L.P., 599 LEXINGTON AVENUE, 40TH FLOOR, NEW YORK, NY, 10022, US

(21) Appl. No.: 09/873,952

(22) Filed: Jun. 4, 2001

(30) Foreign Application Priority Data
Dec. 6, 1998 (DE) 198 55 953.4

(51) Int. Cl. A61K 31/685; A61K 9/127

(52) U.S. Cl. 514/78; 424/450

(57) ABSTRACT

A composition containing an antineoplastic alkylphospholipid, and an antineoplastic antiestrogen in a lipid vesicle; more particularly, the composition is a liposome composition which comprises (i) an antineoplastic alkylphospholipid,(ii) an antineoplastic water- or lipid-soluble antiestrogen associated in liposomal form with the antineoplastic alkylphospholipid, (iii) a nonneoplastic phospholipid, and optionally one or more of (iv) a sterol, (v) a positively or negatively charged lipid, and (vi) a PEG lipid.

### FIELD OF THE INVENTION

The present invention relates to a pharmaceutical composition of an antiestrogen, alkylphospholipids and phospholipids, its manufacture and use.

### BACKGROUND

In tumor drug therapy, optimal treatment is repeatedly inhibited by the occurrence of resistance against the drug and by toxic side effects. Some of these undesired effects can be eliminated or reduced by encapsulation of the drugs in liposomes (D. D. Lasic and D. Papahadjopoulos, Medical Applications of Liposomes, Elsevier, 1998). Liposomal anthracyclins have been employed in numerous clinical applications. Specific benefits result if phospholipids with an inherent antitumor effect are used to form the liposomes, e.g. alkyl phospholipids (Arndt et al. Drugs of Today 1998, 34, 83-96).

Alkyl phospholipids are relatively new type of compounds, the effects of which on tumor growth is achieved by their effects on the cell membrane (Alkylphosphocholines: An update, Drugs of Today, Vol. 34, Suppl. F, 1998). Under certain conditions, alkylphospholipids have supramolecular structures, such as liposomes, with more favorable properties than the monomeric or micellar compound (German patents Nos. 4,132,345 A1; and 4,408,011 C1). Further substances having an antineoplastic effect can also be included in these liposomes that have an antitumor effect (Arndt et al., Breast Cancer Res. Treatm. 43 (1997) 237-246, German patent No. 4,408,011 C1).

Breast cancer is the most frequently occurring tumor in women. It can be influenced in most cases by endocrine measures, as can also other cancers such as of the prostate, uterus, brain, and thyroid cancers. Competitive hormone therapy with tamoxifen is of particular importance in this context; in it, the endogenous hormones are antagonized at the receptor. Treatment with tamoxifen, which has only a few side-effects, is however limited by development of resistance against the drug. The causes of this resistance include alterations of the ligand and its binding to the estrogen receptor (ER), loss or alteration of the ER, alterations of transcription factors or the ER-associated protein or blockage through anti-estrogen binding proteins (Katzenellenbogen et al., Breast Cancer Res. Treat. 44 (1997) 23-38; Osborne, New Engl. J. Med. 339 (1998) 1609-18; U.S. Pat. No. 5,904,930).

### BRIEF DESCRIPTION OF THE INVENTION

It is an object of the present invention to provide an antineoplastic alkylphospholipid in combination with an estrogen in a lipid vesicle (i.e. a liposome) which is effective in antiestrogen resistant tumors and which minimizes or prevents the development of resistance.

The present invention is a pharmaceutical composition which comprises a combination of an antineoplastic alkyl-phospholipid, a water- or lipid-soluble antiestrogen in a lipid vesicle, and a phospholipid, such as phosphatidylcholine, that has no antineoplastic properties. The composition can optionally also include a cholesterol or other sterol, a lipid with a positive or negative charge, and a polyethylene glycol-modified PEG lipid and/or pharmaceutical carriers and/or excipients.

### BRIEF DESCRIPTION OF THE DRAWING

The sole FIGURE of this application shows the cytotoxic effect of tamoxifen liposomes on breast cancer cells.

### DETAILED DESCRIPTION

The essential feature of the invention is a composition which contains an antiineoplastic alkylphospholipid, and an antineoplastic antiestrogen in a lipid vesicle. A suitable example of these ingredients is octadecyl-(N,N-dimethylpiperidin-4-yl)-phosphate (OPP), hexadecylphosphocholine, erucylphosphocholine, octadecylphosphoethanolamine, and hexadecylphosphoserine.

More particularly, the composition of the present invention contains (a) an alkylphospholipid with antineoplastic effect, (b) a water- or lipid-soluble antiestrogen in a lipid vesicle, and (c) an antineoplastically inert phospholipid, and optionally (d) one or more of cholesterol or any other suitable sterol, and a lipid with positive or negative surface charge, and a polyethylene glycol modified lipid (PEG lipid), and further actives as well as a pharmaceutically conventional carrier and/or excipient.

As used herein, “antineoplastically inert” means a compound that has no antineoplastic properties.

The alkylphospholipids of the present composition suitably has the formula

R—Y—P—X (1)

wherein

R is a C₁₂₋₂₂ alkyl, alkenyl or alkinyl residue;

Y is oxygen, sulfur or a CH₂ residue;

P is a phosphate group (PO₂); and

X is a choline, modified choline residue or serine, ethanolamine, glycerine group, or a synthetic modification of the foregoing groups.

Suitable examples of X include hexadecylphosphocholine, octadecylphosphocholine, erucyl-phosphocholine, octadecyl-[2-(N-methylpiperidinio)ethyl]-phosphate, octadecylphospho-ethanolamine and hexadecylphosphoserine. A suitable example of a synthetic modification is the piperidine-4-yl group.

A water or lipid-soluble antiestrogen associated with the phospholipid analogs of Formula (I) is suitably tamoxifen, droloxifene, toremifene, idoxifene, raloxifene, miproxifene-phospate (TAT-59), ICI 1643,384, ICI 182,780 and the main metabolites of tamoxifen, namely 4-hydroxytamoxifen and N-desmethyl-tamoxifen.

Antineoplastically inert phospholipids without their own antineoplastic effect are generally lipids from natural sources or of synthetic origin such as are customarily used for liposome production, for example phosphatidylcholine.

Suitably polyethylene glycol modified phosphatidylethanolamine in the molecular weight range of 1000-6000 Dalton is used as a PEG lipid. For example, suitable compounds include 1,2-distearoyl-s,n-glycero-3-phosphoethanolamine-N-polyethylenglycol, MG2700; (PEG₂₀₀₀DSPE) and 1,2-dipalmitoyl-sn-glycero-3-phosphoethanolamine-N-polyethylenglycol, MG5750 (PEG₅₀₀₀DPPE). Compounds which are simultaneously a PEG lipid and an anti-neoplastically effective phospholipid analog, are also useful, such as hexadecylphosphoethanolamine-N-polyethyleneglycol.

According to the invention, suitably an anti-neoplastically inert lipid of a natural or synthetic origin is used as a base lipid for the membrane formation, such as phosphocholine, serine, ethanolamine, glycerol or other similar lipids, with the ratio of lipid to antiestrogen being from 0 to 10:1 (mass ratio m/m).

Suitably, cholesterol or another suitable sterol such as sitosterol is used with the sterol being in a mol ratio of from 0 to 1:1 to the alkylphospholipid. The liposomal form is suitably a single-layered or multilayered vesicle or the liposomes are available as a reverse evaporation vesicle.

The effect of the agent to overcome resistance according to the present invention can be shown both in vitro and in vivo. The composition of the present invention is pharmaceutically stable, physiologically outstandingly tolerable, and is particularly suited for intravenous application. Undesired metabolism of the antiestrogens is avoided or reduced, and improved resorption and distribution of the drug is achieved. Antiestrogens that are difficult to dissolve in water can be easily applied in a liposomal form. The composition of the present invention is therefore very well suited for application in tumor therapy.

The invention is further illustrated through the following examples.

### EXAMPLE 1

4.62 mg octadecyl-(1,1-dimethyl-piperidino-4-yl)-phosphate (OPP; 10 μmol), 0.387 mg Z-4-hydroxy-tamoxifen (HO-Tam, 1 μmol), 1.55 mg cholesterol (4 μmol), and 1.1 mg dicetylphosphate (DCP; 2 μmol) are completely dissolved in 25 ml chloroform/methanol (7/3; v/v) and the solvent is then completely evaporated on a rotation evaporator. The finely distributed lipid film that is obtained is resuspended with 1 ml of phosphate-buffered salt solution (PBS, pH 7.4) and intensively moved for at least 3 hours at room temperature on a vibration machine following addition of some glass pearls. The resulting suspension of multilayered vesicles (MLV) is then repeatedly extruded through polycarbonate filters of a pore diameter of 100 nm, with a LiposoFast basic system (sold by Avestin, Inc. Ottawa, Canada) until vesicles with an average diameter around 100 nm with a unimodal distribution of sizes and a polydispersity index of less than 0.2 (as determined by Dynamic Light Scatter Measurement, DLS) are obtained.

The content of OPP, HO-Tam, CH and DCP is checked by HPTLC. Over 85% of the original amount is retained. The composition of the liposomes is unchanged compared with the original composition (deviation<5%). These HO-Tam liposomes are most suitably used for in vitro tests.

### EXAMPLE 2

36 mg OPP, 72 mg tamoxifen citrate (Tam), 144 mg phosphatidylcholin (PC) and 8.5 mg DCP are completely dissolved in 100 ml chloroform/methanol (7/3; v/v) and the solvent then completely evaporated on a rotation evaporator. The resulting finely distributed lipid film is resuspended with 12 ml citric acid/phosphate buffer (pH 6.08), and intensively moved for at least 3 hours at room temperature on a vibration machine following addition of some glass pearls. An MLV suspension is obtained, which is heterogeneous and in its size distribution has vesicle diameters of between 100 and 5000 nm.

These Tam liposomes are most suitably used for in vitro tests and as initial liposomes for vesicles of a defined size.

### EXAMPLE 3

36 mg OPP, 72 mg tamoxifen citrate (Tam), 144 mg phosphatidylcholine (PC) and 8.5 mg DCP and 9.7 mg N-(O-methyl-polyethylenglycyl)-1,2-distearyl-s,n-glycero-3- phosphoethanolamine (PEG₂₀₀₀DSPE) are completely dissolved in 100 ml chloroform/methanol (7/3; v/v) and the solvent then completely evaporated on a rotation evaporator. The resulting finely distributed lipid film is resuspended with 12 ml of citric acid/phosphate buffer (pH 6.08) and intensively moved for at least 3 hours at room temperature on a vibration machine following addition of some glass pearls. An MLV suspension is obtained, which is heterogeneous in its size distribution has vesicle diameters of between 100 and 5000 nm. These Tam liposomes are most suitably used for in vitro tests and as initial liposomes for vesicles of a defined composition.

### EXAMPLE 4

Tam MLV's from Example 2 are repeatedly extruded through polycarbonate filters, pore diameter 200 nm, with a LiposoFast basic system (Avestin, Inc. Ottawa, Canada) until a unimodal size distribution around 180 nm is achieved with a poly-dispersity index of less than 0.35 (Dynamic Light Scatter Measurement, DLS).

The content of OPP, Tam, CH and DCP is checked by HPTLC. A liposome suspension containing about 75% of used Tam and 98% of OPP is obtained. In addition, the composition of the liposomes is unchanged compared to the original composition (deviation<5%). These Tam liposomes are most suitably used for in vivo tests.

### EXAMPLE 5

Peg-Tam MLV's from Example 3 are repeatedly extruded through polycarbonate filters, pore diameter 200 nm, with a LiposoFast basic system (Avestin, Inc. Ottawa, Canada) until a unimodal size distribution around 185 nm is achieved with a poly-dispersity index of less than 0.33 (Dynamic Light Scatter Measurement, DLS). The content of OPP, Tam, DCP und Peg₂₀₀₀DSPE is checked with HPTLC. A liposome suspension containing about 75% of used Tam and 98% of OPP is obtained. In addition, the composition of the liposomes is unchanged compared with the original composition (deviation<5%). The Peg-Tam liposomes are most suitably used for in vivo tests.

### EXAMPLE 6

HO-Tam liposomes from Example 1 are diluted with an RPMI medium with 10% fetal calves' serum (without added indicator, with adriamycin/streptomycin) so that a concentration of 200 nmol/ml of OPP is reached, then further serially diluted down to 0.78 nmol/ml. The concentration of HO-Tam active agent is then accordingly from 20 nmol/ml to 0.08 nmol/ml.

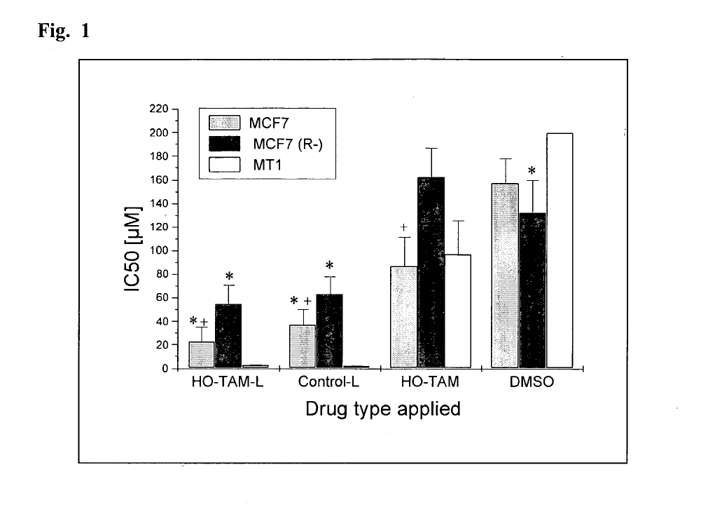

Breast cancer cells MCF7, which are sensitive tamoxifen, and MCF7-R, which are resistant to antiestrogen, are seeded into 96-well plates with a density of 2×10⁴ cells/well and incubated on the following day with HO-Tam liposomes, control liposomes of the composition of the HO-Tam liposomes, but without HO-Tam, HO-Tam dissolved in DMSO and DMSO of the same amount as needed to dissolve the HO-Tam, for three days. The supernatants are then removed, the cells washed with PBS and then the cell growth inhibition determined with the MTT assay. The cells are incubated for this with 200 μl MTT solution (4,6-dimethylthiozol-2-yl-2,5-diphenyl-tetrazolium; 0.5 mg/ml) for 4 hours at 37° C., 170 μl of the supernatant is carefully removed and the precipitated formasan crystals completely dissolved with a 70% isopropyl alcohol solution by intensive pipetting and shaking. After this, the 96-well plates are photospectroscopically measured at 540 nm and the growth inhibition calculated in comparison to the growth of untreated cells. A growth inhibition as portrayed in FIG. 1 is obtained.

### EXAMPLE 7

1×10⁵ cells/ml were incubated with the corresponding liposomes (L), HO-TAM/DMSO and with DMSO for 3 days. The living cells were determined with the MTT assay. The concentration of active agent necessary to inhibit the cell growth by 50% (IC₅₀) is stated.

Tam liposomes according to Example 4 are used for the in vivo treatment test. As a tumor model, breast cancer 3366/Tam is transplanted onto female NMRI nude mice and the treatment started when the tumor is palpable. The animals are given one dose of liposomes with 50 mg/kg Tam (and correspondingly 25 mg/kg OPP) twice a day for 4 weeks. As controls, liposomes containing no Tam are administered, in addition one group being treated with free Tam. The tumor growth in relation to the control group (physiological salt solution) is determined and portrayed as a percentage T/C, as shown in FIG. 1 and in Table 1. The example of FIG. 1 shows that 1×10⁵ cells/ml were incubated with the corresponding liposomes (L), HO-TAM/DMSO and with DMSO for 3 days. The living cells were determined with the MTT assay. The concentration of active agent necessary to inhibit the cell growth by 50% (IC₅₀) is represented. The asterisk * means that the result is significantly different from HO-TAM; and a plus sign + means that the r4esult is: significantly different from MCF7(R−).

TABLE 1
Therapeutic effectivity of tamoxifen liposomes compared with the
resistant breast cancer tumor 3366/Tam

| | | Dose, | Alteration of | |
|---|---|---|---|---|
| | | Tam/Lipid | body weight | T/C |
| Group | Substance | mg/kg/injection | % (day 29/51) | % |
| A | Solvent | | 3 | |
| B | tamoxifen | 50/0 | −5 | 91 |
| C | tamoxifen liposomes | 50/25 | −5 | 63* |
| D | control liposomes | 0/25 | −4 | 88 |

*Significantly different from Tamoxifen and the solvent control (p < 0.05)

We claim:

1. Means for tumor therapy on a liposome basis, containing:
an alkylphospholipid with anti-neoplastic effectivity
a water or lipid-soluble anti-oestrogen with anti-neoplastic effectivity, associated in a liposomal form with the alkylphospholipid
a phospholipid without anti-neoplastic effectivity
if need be, cholesterol or any other suitable sterol
if need be, a lipid with positive or negative charge
if need be, a polyethylene glycol modified lipid (PEG lipid)
pharmaceutically customary carrier and ancillary materials.

2. Means according to claim 1, wherein compounds of general structure 1 are used as alkylphospholipids,
R—Y—P—X Structure I:
with the meanings:
R: an alkyl, alkenyl or alkinyl residue with 12 to 22 C atoms
Y: oxygen, sulphur or CH₂
P: phosphate (PO₂)
X: a choline or modified choline rest or serine, ethanolamine, glycerine groups or synthetic modifications of these groups such as the piperidine-4-yl group

3. Means according to claims 1 and 2, wherein suitably hexadecylphosphocholine, octadecylphosphocholine, erucylphosphocholine, octadecyl- [2-(N-methylpiperidino)ethyl]phosphate, octadecylphosphoethanolamine and hexadecyl-phosphoserine are used.

4. Means according to claim 1, wherein anti-oestrogens such as Tamoxifen, Droloxifen, Toremifene, Idoxifene, Raloxifene, Miproxifene-Phospate (TAT-59), ICI 164,3384, ICI 182,780 and the main metabolites of Tamoxifen, 4-hydroxytamoxifen, N-desmethyltamoxifen, including new anti-oestrogens not yet described in more detail, are used as further substances with an anti-neoplastic effect, associated with the alkylphospholipid in a liposomal form.

5. Means according to claim 1, wherein an anti-neoplastically inert lipid of natural or synthetic origin such as phosphocholine, serine, ethanolamine, glycerol or other similar lipids is used as the basic lipid for the membrane formation, the ratio of lipid to anti-oestrogen being 0-10:1 (m/m).

6. Means according to claim 1, wherein cholesterol or another suitable sterol such as sitosterol is contained and the sterol is in a molar ratio of 0-1:1 to the alkylphospholipid.

7. Means according to claim 1, wherein a polyethylene glycol modified lipid (PEG-Lipid), suitably N-(O-methoxy-polyethylenglycyl)-1,2-distearyl-s,n-glycero-3-phosphoethanolamine (PEG₂₀₀₀-DSPE) is added.

8. Means according to claim 1, wherein it contains OPP as the first anti-neoplastically effective substance and Tamoxifen as a further anti-neoplastically effective substance.
